(19) United States
(12) Patent Application Publication
(10) Pub. No.: US 2009/0264785 A1
(43) Pub. Date: Oct. 22, 2009

(54) Method and Apparatus For Assessing Brain Function Using Diffusion Geometric Analysis

(75) Inventors: Elvir Causevic, New York, NY (US); Ronald Coifman, North Haven, CT (US); Andreas Coppi, Groton, CT (US); Frederick Warner, New Haven, CT (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BrainScope Company, Inc.

(21) Appl. No.: 12/105,439

(22) Filed: Apr. 18, 2008

(51) Int. Cl. A61B 5/0402

(52) U.S. Cl. 600/544

(57) ABSTRACT

A method of extracting features and classifying a neurological state of a subject is provided. The method includes recording brain electrical activity, organizing the recorded data set into digital documents, computing a diffusion geometry on the data set comprising at least a plurality of diffusion coordinates, and classifying the data set into a neurological state based on the metrics provided by the diffusion coordinates.

### FIELD OF THE INVENTION

This invention relates to the field of neurological evaluation using brain electrical activity, and more specifically, to the method and apparatus for automatic, on-site assessment of brain function using diffusion geometric analysis of recorded brain electrical activity.

### BACKGROUND OF THE INVENTION

The central nervous system (CNS) and the brain in particular, perform the most complex and essential processes in the human body. Surprisingly, contemporary healthcare lacks sophisticated tools to objectively assess their function. A patient's mental and neurological status is typically assessed clinically by an interview and a subjective physical exam. The clinical laboratory currently has no capacity to assess brain function or pathology, contributing little more than identification of poisons, toxins, or drugs that may have externally impacted the CNS. Brain imaging studies, such as computed tomography imaging (CT), magnetic resonance imaging (MRI), though widely used and useful, are structural/anatomical tests revealing little or nothing about brain function. In the immediate time of acute brain injury, stroke, or seizure, imaging studies typically reveal no abnormality, even when there is clear and dramatically abnormal brain function. CT and MRI only detect the condition after the morphology or structure of the brain has changed. In some cases it can take from hours to days after the patient is present in an emergency room (ER) before overt changes are evident on the CT or MRI, and before severe neurological pathology is visible. Electrical activity of the brain, however, is affected immediately. New imaging modalities such as functional MRI (fMRI) measure the changes in oxygen saturation in different parts of the brain. Radioisotope imaging such as positron emission tomography (PET) and single photon emission computerized tomography (SPECT) assess chemical changes within the brain as a measurement of function with limited sensitivity and specificity. All of these assessment tools play an important role in selected cases, but they are costly, not universally available, and they do not provide critical information at the early stages of acute care situations. None of the current techniques provides the immediate, actionable information critical to timely intervention, appropriate triage, or the formulation of an appropriate plan of care.

The CNS and brain, of all organs in the human body, are also the most time sensitive and have the least capacity for repair. Currently, emergency room patients with altered mental status, acute neuropathy, or head trauma must undergo costly and time consuming tests to determine an appropriate course of treatment. Unfortunately, in many cases, the clinical condition of patients continue to deteriorate as they wait for equipment to become available or for specialists to interpret tests. The task of the ER physician is to basically establish whether the brain is functioning normally, whether the abnormality is psychiatric or organic in nature, whether an organic abnormality is global or lateralized, and to develop an initial assessment of the diagnostic possibilities. The problem that faces ER physicians is that their resources are quite literally limited to a flashlight and a rubber reflex hammer. Amazingly, all of the physician's decisions concerning the administration of emergency treatment or intervention, including CT scan, spinal tap, additional consultation or discharge are based on the results of this simplistic exam.

Often, ER patients are sent for imaging studies, yet many functional brain abnormalities, such as seizure, are not visible on a CT scan. Some abnormalities which will eventually have anatomical and structural consequences often take time to become visible. This is true for many important conditions such as ischemic stroke, concussion, raised intracranial pressure, and others. Thus, while the location, expense, and limited availability of the CT scan can be problematic, so indeed can the fact that it is a structural as opposed to functional test.

One-third of over 200 physicians surveyed at the American College of Emergency Physicians feel that the combination of a good clinical laboratory, a neurological exam, and a CT scan of the head, is not adequate for the assessment of every patient with altered mental status or neurological dysfunction. Consensus estimates from the CDC NHS database and practicing ER physicians is that patients requiring a mental status exam represent 15% of the more than 100 million ER visits annually in the U.S., and in some institutions, considerably more.

There are more than 100 million ER visits per year in the US alone (CDC/NCHS) database. In the year 2000, more than 13 million of these patients required a formal mental status exam and nearly 5 million had CT scans. This data indicates the need for real-time functional brain state assessment which can be performed in the hospital, in an ambulance, at a sporting event, or any other location where acute neurological evaluation may be necessary.

All of the brain's activity, whether reflexive, automatic, unconscious, or conscious, is electrical in nature. Through a series of electro-chemical reactions, mediated by molecules called neurotransmitters, electrical potentials (voltages) are generated and transmitted throughout the brain, traveling continuously between and among the myriad of neurons. This activity establishes the basic electrical signatures of the electroencephalogram (EEG) and creates identifiable frequencies which have a basis in anatomic structure and function. Understanding these basic rhythms and their significance makes it possible to characterize the EEG as being within or beyond normal limits. At this basic level, the EEG serves as a signature for both normal and abnormal brain function.

The electrical activity of the brain has been studied extensively since the first recordings over 75 years ago, and especially since the advent of computers. “Normal” electrical activity of the brain has been well characterized in hundreds of studies, with a narrow standard deviation. The frequencies of electrical activity of some parts of the brain are the normal response to various stimuli, such as acoustic, visual, or pain, known as “evoked potentials.” Evoked potentials (EP) are particular waves that have characteristic shapes, amplitudes and duration of peaks within those wave shapes, and many other features, all of which have well established normative data, generated over decades of research. Normative data for all of the recorded brain electrical activity are remarkably constant across different genders, ages, and ethnicities. Moreover, any variability that does exist is well described and explained.

Neuroscientists have also characterized the brain electrical activity signature of various different brain pathologies. Just as an abnormal electrocardiogram (ECG) pattern is a strong indication of a particular heart pathology, an irregular brain wave pattern is a strong indication of a particular brain pathology. A wide array of pathologies have been well characterized: acute and chronic, structural, toxic, metabolic, and even specific diagnoses such as: ischemic stroke, epileptic seizures, concussion, alcohol, and drug overdose, psychiatric conditions, and dementias including Alzheimer's disease. A large body of data, with continuing refinements and contributions, constitutes the field of clinical neurophysiology.

Even though neurometric technology is accepted today and a tremendous body of data exists, its application in the clinical environment is notably limited. Some of the barriers limiting its adoption include: the cost of typical equipment for obtaining and analyzing brain electrical activity, the lack of portability of the equipment, the need for a technician to administer the test, the time it takes to conduct the test, and the need for expert interpretation of the raw data. More importantly, the technology is neither available nor practical in the acute care setting, especially at the point of care. A complete diagnostic EEG instrument, fully equipped, typically costs $80,000. Despite the high costs, the instrument produces essentially raw waveforms which must be carefully interpreted by an expert. Moreover, use of the standard EEG equipment remains extremely cumbersome. It can take 30 minutes or more to apply the required 19 electrodes. Once the patient is prepared for the test, the recording itself can take from 1 to 4 hours. Data is collected and analyzed by an EEG technician, and are then presented to a neurologist for interpretation and clinical assessment. There are some self-standing dedicated neurodiagnostic laboratories, which focus strictly on detailed analysis of electrical brain data. Neither the specialized centers, nor the typically large hospital EEG machines are practical for the ER, operating room (OR), intensive care unit (ICU), or any other acute care setting where patients are in the greatest need. Immediate, functional brain state assessment is needed to treat patients with acute neurological injury and disease for the prevention of further damage and disability. The recently developed neurological evaluation system using BrainScope Bx™ technology provides the immediate functional brain state assessment that is needed to treat such acute neurological injuries. This system is described in U.S. patent application Ser. No. 11/195,001 (“the '001 application”), which is herein incorporated by reference in its entirety. The application of diffusion geometric analysis to the proprietary Bx™ technology, as described herein, enables quicker and more accurate diagnosis of the neurological state of a subject.

### SUMMARY OF THE INVENTION

The '001 application provides a method which is fully capable of analyzing recorded brain electrical activity, and providing a functional brain state assessment in a portable device. The neurological evaluation process, however, can be further enhanced if the feature extraction and classification processes are performed using diffusion geometry-based signal processing techniques, as described herein, which enables the definition of affinities and related scales between any digital data points, to be used for noise removal, and as features in classification, automatic model building, and similar tasks. The diffusion geometric analysis can be used to consider a recorded brain electrical activity dataset as a collection of digital documents, and augmenting the similarity or nearness concepts or measures between these documents using empirically derived diffusion geometries, as further defined and described herein.

In accordance with the invention, there is provided a method and a system for recording brain electrical activity and analyzing the recorded data set using diffusion geometric analysis. In an exemplary embodiment consistent with the present invention, the recorded brain electrical activity may be considered as a collection of data objects, for which there is at least some rudimentary notion of similarity, closeness, or nearness of at least two of the individual data objects. However, for sorting high-dimensional data, such as recorded brain electrical activity, certain standard notions of similarity or nearness (e.g. conventional Euclidean metrics) are not very useful inference tools. As such, in the exemplary embodiment using Bx™ technology, there is provided a technique, wherein the data objects may be automatically re-mapped into a low-dimensional embedding, so that ordinary Euclidean metrics become more useful and relevant. This may be accomplished with empirically derived diffusion geometries.

It is not typically practical to compute or use diffusion distances of high-dimensional data. This is generally because standard computations of the diffusion metric require d*n² or d*n³ number of computations, d being the dimension of the data, and n being the number of data points. This would be expected because there are O(n²) pairs of data points, and thus n² operations would typically need to be performed in order to compute all pairwise “distances”. However, approximations to these distances can be computed in fixed linear time O(n) or O(n log(n)), to within any desired precision. A method for computing data sets in this manner is fully disclosed in U.S. patent application Ser. No. 11/165,633 (“the '633 application”), which is herein incorporated by reference in its entirety.

The method disclosed in the '633 application describes a natural data-driven self-induced multiscale organization of data in which different time/scale parameters correspond to different representations of the data structure at different levels of granularity, while preserving microscopic similarity relations. Based on this method, recorded brain activity can be analyzed to determine numerous measures of distance, similarity, etc, which can be assigned a quantitative measure. Using such a method, brain electrical activity records, or their extracted features, may be thought of as a collection of digital documents, and the documents as an ordered list of numbers (coordinates) representing parametric attributes of the document. Such documents originating from brain data may have dimensions exceeding 1000, such that the given metrics, (i.e. the notions of similarity, etc.) represent only very strong similarities between the documents. Such similarity relations are then extended to documents that are not directly and obviously related by analyzing all possible chains of links or similarities connecting them. This may be achieved through the use of diffusion processes, and this leads to a very simple and robust quantity that can be measured as an ordinary Euclidean distance in a low dimensional embedding of the data. Further, brain electrical data contaminated by noise may be thought of as a collection of digital documents, and the features of brain signals can be separated from the noise that contaminates it using the notions of affinity, distance, nearness, similarity, etc.

Consistent with the present invention, there is provided a method of extracting features and classifying a neurological state of a subject, comprising measuring time-series features, organizing the features into data sets, computing diffusion geometry on the data sets, and classifying the data sets into neurological states.

Consistent with the present invention, there is also provided a method for obtaining a database of data relating to brain activity, comprising measuring signals related to brain activity, organizing the signals into data sets, computing diffusion geometry on the data sets to produce data sets having associated diffusion coordinates, and storing the diffusion coordinates in the database.

Further consistent with the present invention, there is provided a method for filtering or denoising signals representative of a subject's brain activity, comprising measuring the signals, organizing the signals into data sets, computing diffusion geometry on the data sets, and filtering or denoising the signals according to the diffusion coordinates of the data sets.

Further consistent with the present invention, there is also provided a device for classifying a neurological state of a subject, comprising a processor, and a memory, the memory containing instructions for execution by the processor for performing a classification routine, the classification routine comprising measuring time-series features, organizing the features into data sets, computing diffusion geometry on the data sets, and classifying the diffused data sets into neurological states.

Additional features and advantages of the invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiment of the invention and together with the description, serve to explain the principles of the invention.

### BRIEF DESCRIPTION OF THE DRAWINGS

FIG. 1 is a flowchart illustrating a method for classifying a neurological state of a subject, consistent with the present invention.

FIG. 2 is a graph illustrating potential vectors forming clusters when plotted in diffusion coordinates space.

FIG. 3 illustrates a tabulation in three dimension of diffusion coordinates of all possible 20-electrode measurements.

FIG. 4 is a graph illustrating the principal left singular vector plotted in its diffusion geometry coordinates space.

FIG. 5 is a graph illustrating the principal right singular vector plotted in its diffusion geometry coordinates space.

FIG. 6 is a flowchart illustrating a method for determining a probability for transitioning between states, consistent with the present invention.

FIG. 7 shows a portable handheld base unit of a device for determining a neurological state of a subject, consistent with the present invention.

FIG. 8 shows a schematic diagram of the portable handheld base unit, consistent with the present invention.

FIG. 9 is a diagram illustrating an electrode set according to an embodiment consistent with the present invention.

### DESCRIPTION OF THE ILLUSTRATIVE EMBODIMENTS

Reference will now be made in detail to the present embodiments (exemplary embodiments) of the invention, examples of which are illustrated in the accompanying drawings. Wherever possible, the same reference numbers will be used throughout the drawings to refer to the same or like parts.

Using Bx™ technology, collected normative data has been used to establish quantitative features of brain electrical activity which clearly distinguish normal brain function from abnormal dysfunctional conditions. This normative data has been shown to be independent of racial background and to have extremely high test-retest reliability, specificity (low false positive rate) and sensitivity (low false negative rate). Conducted studies of 15,000 normal and pathological evaluations have demonstrated that brain electrical signals are highly sensitive to changes in normal brain function, and change their characteristics instantaneously after catastrophic events such as concussive (blast) or traumatic (impact) brain injuries, ischemia or stroke, and also reflect a variety of chronic developmental, neurological and psychiatric disorders which are not related to any detectable change in physical brain structure. Because different types of brain injuries and diseases affect brain electrical activity in different ways, it is possible to differentiate not only normal from abnormal function, but also to independently determine which kind of pathology is affecting the brain and to what degree, providing guidance on how to restore brain function toward more normal operation. Embodiments consistent with the present invention use this as a basis for providing a diagnosis. Moreover, embodiments consistent with the present invention may utilize the methods disclosed herein for performing a diffusion geometry analysis on the pre-acquired brain electrical activity records to provide points for comparison for the brain electrical activity records obtained from the subject, and/or used to model brain activity and neurological states.

The methods disclosed herein provide a framework for structural multiscale geometric harmonic analysis on brain electrical activity records. Diffusion maps are used to generate multiscale geometries in order to organize and represent complex structures. Appropriately selected eigenfunctions of Markov matrices (describing local transitions, inferences, or affinities in the system) may lead to macroscopic organization of the data in the brain electrical activity records at different scales. In particular, the top of such eigenfunctions may be the coordinates of the diffusion map embedding.

A diffusion map may generally be constructed given any measure space of points X and any appropriate kernel k(x,y) describing a relationship between points x and y lying in X. The methods and algorithms disclosed herein may act on a set X of data, with n points, with the values of X being the initial coordinates of the brain electrical activity records. Some of the algorithms disclosed herein have been disclosed in the '633 application, which is herein incorporated by reference in its entirety. However, the algorithms, as used herein, have been adapted to compute diffusion geometry coordinates specifically on brain electrical activity records. In particular, diffusion geometries have been computed on the collected normative data of more than 15,000 normal and pathological evaluations, as described above, and may be computed on brain electrical activity of a subject using a device consistent with the present invention.

In general, the algorithm for computing a diffusion geometry includes the following as inputs: an n×n matrix T, wherein the value T(x,y) measures the similarity between data elements x and y in X; an optional threshold parameter ε with a default of ε=0, used to denoise T; an optional output dimension, with a default of k=n as the desired dimension of the output dataspace. The algorithm will output an n×k matrix A, wherein the value A(n₀,−) gives the coordinates of the n₀th point, embedded into k-dimensional space, at time t=1; and a sequence of eigenvalues λ₁, . . . ,λₖ. An algorithm consistent with present invention is as follows: first, set T₁(x,y)=T(x,y) if |T(x,y)|>ε, with T₁(x,y)=0 otherwise. Next, set λ₁, . . . ,λₖ equal to the largest k eigenvalues of T₁. Then set A to the matrix, the columns of which are the eigenvectors of T₁ corresponding to the largest k eigenvalues of T₁.

Based on the algorithm, the diffusion coordinates at time t, diffCoordₜ(x) may be computed based on the following equation:

diffCoordₜ(x)=[λᵢ^{t}A(x,i)]_{i=1, . . . ,k}

The diffusion distance at time t, dₜ(x,y) may then be computed via the Euclidean distance on the diffusion coordinates using the following equation, adapted from the basic equation for computing a distance between two points in Euclidean space:

$${{d}_{t} ( x , y )}^{2} = {\sum }_{i = 1}^{k} {{λ}_{i}^{2 t} ( A ( x , i ) - A ( y , i ) )}^{2}$$

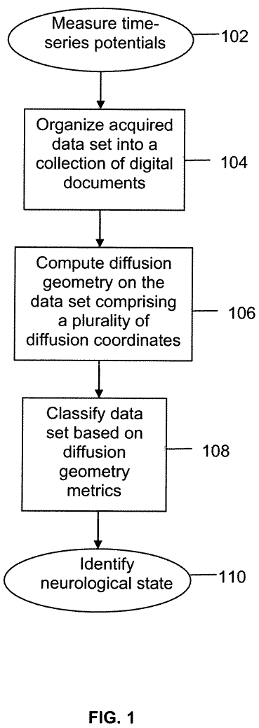

FIG. 1 is a flowchart illustrating a basic method feature extraction and for classifying a neurological state of a subject, using diffusion geometries, consistent with the present invention. The method as disclosed herein may be implemented as a coded algorithm stored in a memory and executed by a processor of a device for classifying a neurological state of a subject, consistent with the present invention. The device is ideally a neurological evaluation system that utilizes the BrainScope Bx™ technology, as disclosed in the '001 application which is incorporated by reference herein in its entirety, but may also be any other device having a memory and a processor, and capable of monitoring brain electrical activity.

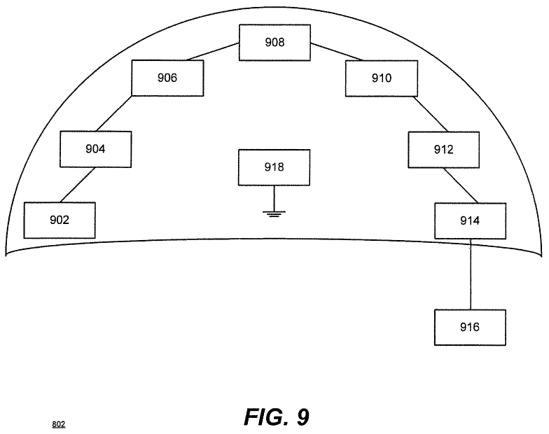

As shown in FIG. 1, time-series potentials are measured (step 102). The time-series potentials may include brain electrical activity over time, and may be measured using standard electrodes, as known in the art, or may be measured using a reduced electrode set as shown in FIG. 9, and further discussed below. Consistent with the present invention, other spacio-temporal features may also be measured. The temporal features may include features of wavelet packets, and the spatial features comprise locations of electrodes, which may be placed on a subject to acquire brain electrical activity records. Further consistent with the present invention, the temporal features may further include spontaneous brain electrical activity, and evoked potentials generated in response to applied stimuli.

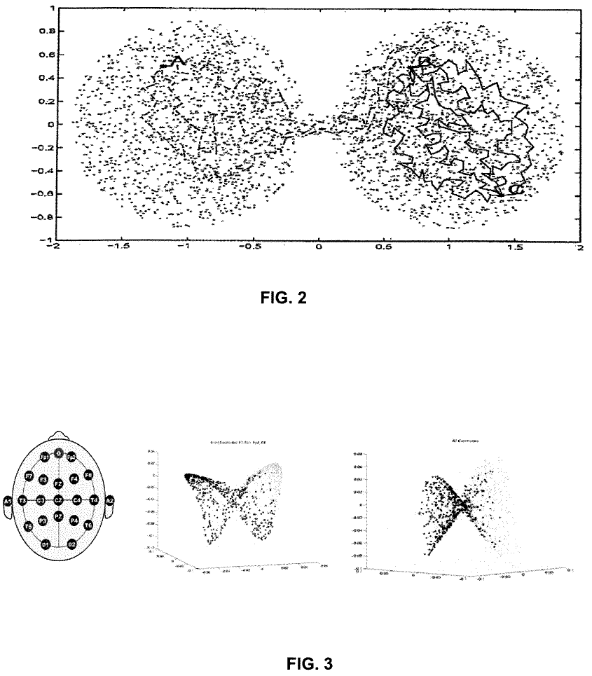

Returning to FIG. 1, the features may then be organized into a plurality of digital documents (step 104), each including a time window of temporal features of the measurement of each electrode, wherein such time features could consist of a band pass filter or wavelet packet feature of the measurement. A diffusion geometry is then computed (step 106) comprising a plurality of diffusion distances of the plurality of documents. The diffusion geometry may be computed using the algorithms described herein, and in the '633 application, but may be computed using any known algorithm capable of achieving a diffusion geometry. The data set is then classified based on the metrics provided by the diffusion geometry (step 108), and a neurological state is identified based on the classification (step 110). The data set may be classified by a number of different criteria. In one embodiment consistent with the present invention, predetermined portions of the diffusion coordinates space may be partitioned into partitions corresponding to particular neurological states. In another embodiment consistent with the present invention, the data set may be classified according to a Euclidean distance between documents, as computed in the diffusion geometry. Moreover, in another embodiment consistent with the present invention, computing a diffusion geometry on the plurality of digital documents may result in clusters in the multi-scale structure (see, for example, FIG. 2). The clusters may represent specific classifications, depending on the metrics used to initialize the cluster. Each metric in the multi-scale structure corresponds to one of the diffusion distances of the plurality of digital documents. A cluster is at first initialized based on one metric, and then hierarchically aggregated based on a different metric from the multiplicity of metrics corresponding to the diffusion distances. In yet another embodiment, classifying the neurological state comprises the step of comparing a present data set to another stored dataset based on diffusion geometry associated with each dataset.

Consistent with the present invention, there is provided a method of breaking up a brain electrical activity record into a time-stamped list of voltage measurements of a plurality of electrodes. In particular, a recorded data set may be considered as a collection of digital documents, wherein each document is made up of a time window of temporal features of the measurement of each electrode. Specifically, a document stamped by time i may comprise an M×L matrix having the form:

v(i)={v_{i,1}, . . . ,v_{i,L}},

where M is the number of electrode channels and L is the length of the time window, where each column vector is

v_{i,n}=[x₁(tᵢ₊ₙ₋₁),x₂(tᵢ₊ₙ₋₁),x₃(tᵢ₊ₙ₋₁),x₄(tᵢ₊ₙ₋₁), . . . ,x_{M}(tᵢ₊ₙ₋₁)],

and xₖ(tᵢ) is the time series data features for channel k at time label i for a particular brain electrical activity record.

To each document, there is associated a dominant temporal feature consisting in the principal left singular vector Lᵢ, or the dominant electrode feature consisting in principal right singular vector Rᵢ of matrix v(i), which is used to measure affinity between documents. The affinity may be computed using an appropriate affinity matrix A. An affinity matrix A, between a document at time i and a document at time j may be defined as

$${A}_{i , j} = \frac{{e}^{\frac{- {‖ v ( i ) - v ( j ) ‖}^{2}}{ɛ}}}{w ( i ) w ( j )} ,$$

wherein ε is a threshold parameter, w(i) is a weighting function at time i, and w(j) is the weighting function at time j, and the weighting functions are selected such that A is Markov in i and j. Next, the eigenvectors of the affinity matrix A are determined, and the eigenvectors are used to construct a Euclidean space representing the diffusion geometry. Consistent with an embodiment of the present invention, the first few eigenvectors of the affinity matrix A may be used to tabulate, parameterize and organize efficiently an entire collection of documents, providing data compression and organization of the microstates and dynamics of the systems. If the first three eigenvectors are used, we obtain an embedding into three dimensional Euclidean space, in which the Euclidean distances measures the affinity diffusion distances between various documents. This embedding is used to classify microstate configurations. To be specific, the bi Markov matrix A defined above may be represented in terms of its eigenvectors, and then the diffusion map at time t may be defined into m dimensional Euclidean space by

A_{p,q}=Σλₗ²φₗ(Xₚ)φₗ(X_{q}) Xₚ→(λ₁^{t}φ₁(Xₚ),λ₂^{t}φ₂(Xₚ),λ₃^{t}φ₃(Xₚ), . . . ,λₘ^{t}φₘ(Xₚ))=\tilde{X}ₚ^{t}

For a given time t, we determine m so that λₘ₊₁^{t} is negligible. The diffusion distance at time t between Xₚ and X_{q} is given as

dₜ²(p,q)=A_{p,p}+A_{q,q}−2A_{p,q}=Σλₗ^{2t}(φₗ(Xₚ)−φₗ(X_{q}))²=∥\tilde{X}ₚ^{t}−\tilde{X}_{q}^{t}∥²

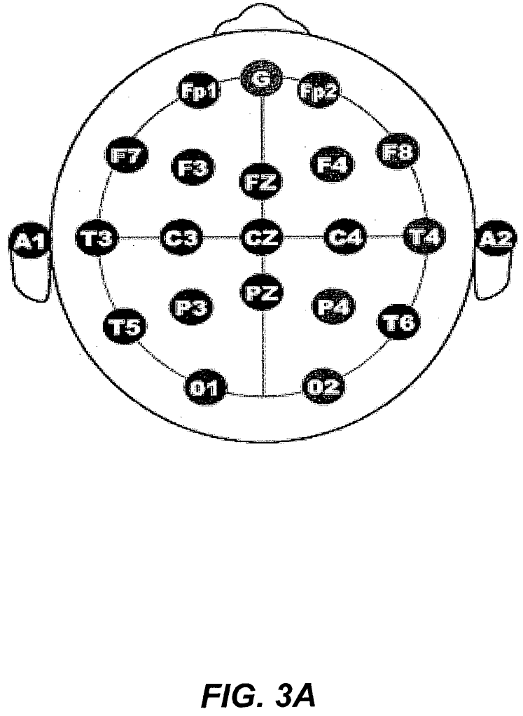

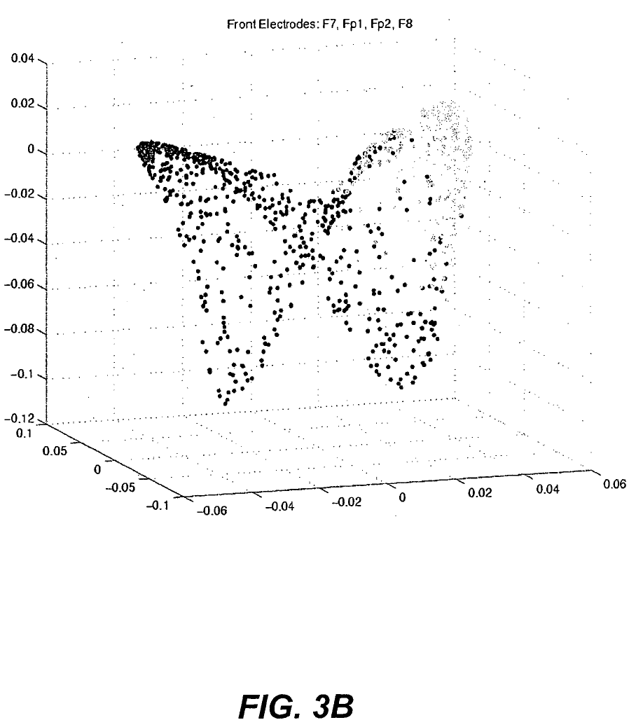

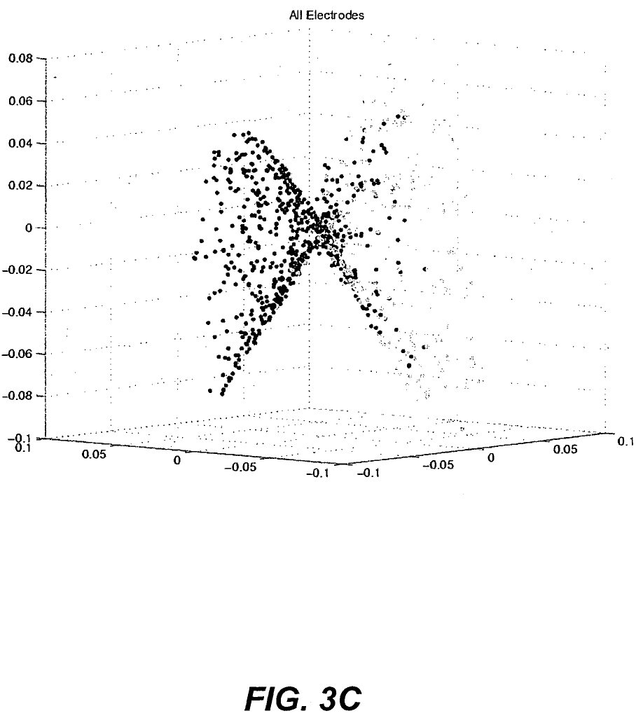

This mapping enables us to represent geometrically an abstract set of measurements on a measurement space, as is illustrated in FIG. 3, which provides a tabulation in three dimensions of all possible 20-electrode measurements. In this case, 20 electrodes measure coherent electrical activity in the brain. Mapping the configuration space of the measurements of 4 electrodes lead to the same configuration as for all 20. In a linear case this would be obtained by decorrelating the outputs, however, here different locations of sources result in different attenuation vectors, or linear decorrelations. Here the first three nontrivial eigenvectors are used to map the data to three dimensions (diffusion map). Thus 4 electrodes may be sufficient to get essentially the same measurements, however, redundancy may be useful in obtaining a cleaner version.

The affinity matrix may also be used to construct a Markov transition probability matrix whose eigenvectors are used to perform clustering. \tilde{L}ᵢ and \tilde{R}ᵢ are defined to be the collection of leading eigenvectors of the affinity matrix. A graph having these vectors as vertices is then formed and the top few eigenfunctions of the Laplacian on this graph is determined, considering these eigenfunctions as new coordinates with which to describe the vectors \tilde{L}ᵢ. The eigenfunctions of the Laplacian for the graph defined from the collection \tilde{R}ᵢ are also determined, wherein the top few such eigenfunctions are retained.

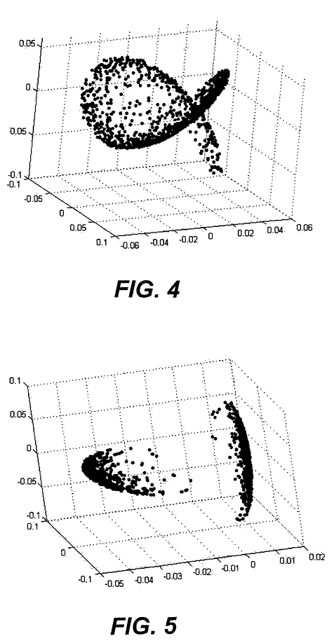

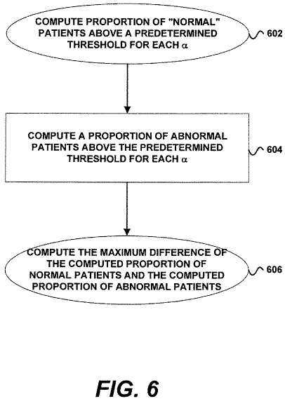

FIGS. 4 and 5 show typical eigenfunctions arising from these constructions. FIG. 5 indicates that there are two spatial “states”, and the state membership can be determined by the sign of one of the eigenfunctions. The pattern of sign changes thus may correspond to the itinerary of the brain's electrical state, which can be studied for possible diagnostic value. For example, the probability of transitioning from a state “1” to a state “2” may be considered, which may be defined as the number of such transitions divided by the total number of transitions (wherein going from a state to itself is considered a transition). For subject j, let fⱼ be this transition probability and consider two subject populations: the “normals”, denoted by Nor, and the “abnormals”, denoted by Abn. The values of fⱼ in separating the Nor and Abn may then be assessed by calculating the maximal probability contrast p(f). To define p(f), consider quantities q(a) of the data fⱼ for all values of a from 0 to 1. For each a, the proportion of normal patients above q(a) (step 602) and the proportion of abnormal patents above q(a) (step 604) may be computed, as follows:

$${p}_{Nor} ( a ) = \frac{1}{\# ( Nor )} {\sum }_{j ∈ Nor} Ind [ {f}_{j} > q ( a ) ]$$

for the normals, and

$${p}_{Abn} ( a ) = \frac{1}{\# ( Abn )} {\sum }_{j ∈ Abn} Ind [ {f}_{j} > q ( a ) ]$$

for the abnormals. Then, p(f) is just the maximum difference of these proportions:

$$p ( f ) = {max}_{a} \vert {p}_{Nor} ( a ) - {p}_{Abn} ( a ) \vert ( step 606 ) .$$

Further consistent with the present invention, diffusion geometry and the general principles behind diffusion analysis may be used to remove noise and artifacts, or “denoise”, signals, including brain electrical activity signals. The diffusion geometry algorithms disclosed herein may further include the processes of the construction of the wavelets at each scale including an orthogonalization step to find an orthonormal basis of functions for the orthogonal complement of the scaling function space at the scale into the scaling function space at the previous scale. The construction of the scaling functions and wavelets allows the analysis of functions on the original graph or manifold in a multiscale fashion, generalizing the classical Euclidean, low-dimensional wavelet transform and related algorithms. In particular, the wavelet transform generalizes to a diffusion wavelet transform, allowing one to encode efficiently functions on the graph in terms of their diffusion wavelet and scaling function coefficients. In some embodiments of the present invention, the wavelet algorithms known to those skilled in the art are practiced with diffusion wavelets as disclosed herein. Moreover, wavelet algorithms are further disclosed in the '001 application, and U.S. Pat. No. 7,302,064, which is incorporated herein by reference in its entirety. For example, functions on the graph or manifold can be compressed and denoised, by generalizing in the obvious way the standard algorithms (e.g. hard or soft wavelet thresholding) for these task based on classical wavelets. For example, if each cluster on a graph (as shown in, for example, FIG. 2) has a number of coordinates, each coordinate is a function on the graph that can be compressed and denoised, and a denoised graph, where each cluster has as coordinates the denoised or compressed coordinates, is obtained. This allows a nonlinear structural multiscale denoising of the whole data set. For example, when applied to a noisy mesh or cloud of points, this results in a denoised mesh or cloud of points. Similarly, diffusion wavelets and scaling functions can be used for regression and learning tasks, for functions on the graph, this task being essentially equivalent to the tasks of compressing and denoising discussed above. As an example, standard regression algorithms known for classical wavelets can be generalized in an obvious way to algorithms working with diffusion wavelets.

However, a simple computationally efficient way to describe the use of diffusion analysis to eliminate noise and artifacts of brain electrical activity signals data or any function on the data, where a function I(i) could describe any attribute or diagnostic quantity (or the actual measured voltage of a given electrode at time i). The data-driven diffusion defined above, may be used to filter the function I(i) as

$$\overline{I} ( i ) = {\sum }_{q} {A}_{i , j} ( j ) = {\sum }_{i} \frac{{e}^{( \frac{- {‖ v ( i ) - v ( j ) ‖}^{2}}{ɛ} )}}{{\sum }_{{j}^{′}} {e}^{( \frac{- {‖ v ( i ) - v ( j ) ‖}^{2}}{ɛ} )}} I ( j )$$

Artifacts and transients can then be extracted by considering the distance between the filtered function and the original. The eigenvectors of the matrix A can also be used to expand the function I(i) to achieve similar signal processing and filtering.

This nonlinear filtering operation compares the values of the function I(i) to other values of that function at other similar brain electrical activity records. This method can also be used to extract evoked responses from large background signals.

An alternate simple data denoising method which is more effective computationally is to use the projection of each electrode data on the left or right singular vector of the matrix v(i), such that the projections could be restricted to the microstate data corresponding to the case that v(i) is of rank one.

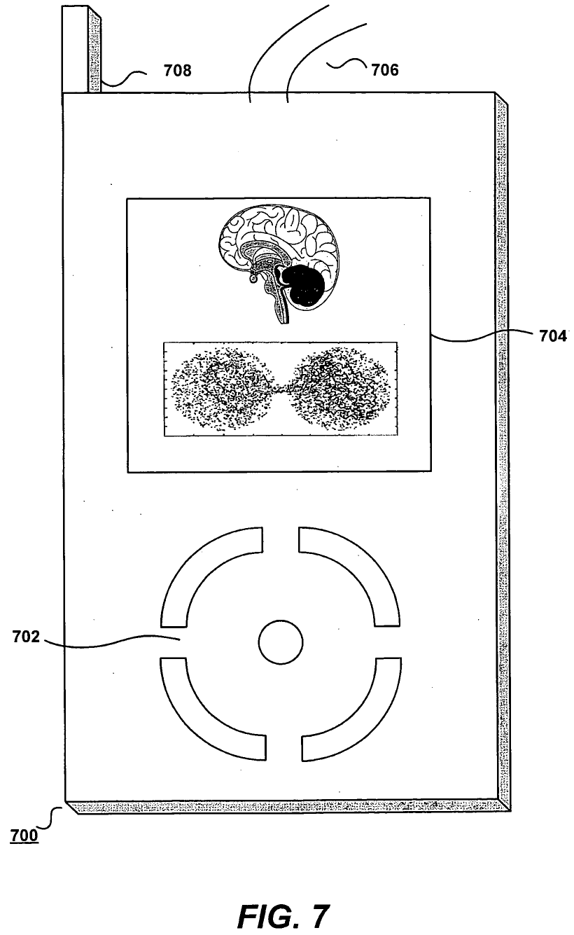

The method as disclosed herein may be implemented as a coded algorithm stored in a memory and executed by a processor of a device for classifying a neurological state of a subject, consistent with the present invention. The device is ideally a neurological evaluation system using Bx™ technology, as disclosed in the '001 application which is incorporated by reference herein in its entirety, but may also be any other device having a memory and a processor, and capable of monitoring brain electrical activity. An example of such a device is shown in FIG. 7. In particular, FIG. 7 shows a portable handheld base unit of a device for determining a neurological state of a subject, consistent with the present invention.

As shown in FIG. 7, the portable handheld base unit 700, in accordance with the Bx™ technology, includes a navigation pad 702, which may include a plurality of navigation buttons and a selection button, allowing a user to navigate through menus illustrated on a screen 704, and select options presented on screen 704. Consistent with the present invention, screen 704 may comprise an LCD, LED, OLED, or plasma screen. Screen 704 may also comprise simple LED (or other illumination means) indicators, which provide an indication of, for example, whether the device is on, if tests are being performed, or the neurological state of a subject.

Consistent with the present invention, navigation pad 702 may be used to select and execute functions to be performed by handheld base unit 700. For example, screen 704 may display a menu of options highlighting possible options for performing tests on a subject. These options may include beginning testing, selecting the types of tests to perform, and/or options for processing or transmitting acquired data.

Handheld base unit 700 may be coupled to a headset, including a plurality of electrodes such as illustrated in FIG. 9, via connecting means 706. Connecting means 706 may include a permanently attached or detachable cable or wire, or may include wireless transceivers, capable of wirelessly transmitting and receiving signals between the headset and the handheld device.

Handheld base unit 700 may also include transceiving antenna 708. Consistent with an embodiment of the present invention, transceiving antenna 708 may be used to wirelessly transmit data stored in the handheld base unit 700 to a remote location for storage or further processing. This data may include diagnosis data, treatment data, or raw electrical signals. The remote location may be a personal computer or a large database. A personal computer may be used for storing and further processing acquired data, allowing, for example, a medical professional to monitor the progress of a subject through the treatment of a concussion. A remote database may be used for storing the acquired data, to allow the acquired data to be added to a larger data pool of subjects having similar brain electrical signals. This larger data pool may be used for neurometric studies to provide a more accurate diagnosis on the basis of comparison.

Further consistent with the present invention, software stored in a memory of handheld base unit 700 may be configured to acquire brain electrical activity records of the subject, and perform diffusion geometric analysis to determine a neurological state of the subject, consistent with the methods disclosed herein. Software stored in a memory of handheld base unit 700 may also be configured to display on screen 704 the results of the testing. Results may include displaying a brain map generated from the acquired data showing an indication of a brain injury, a location of a brain injury, or a severity of a brain injury. Results may also include the neurological state of the subject, or simply the computed diffusion geometry.

Software stored in a memory of handheld base unit 700 may further be configured to display on screen 704 additional information related to the testing of a subject or the operation of the device. For example, memory may contain interactive instructions for using and operating the device to be displayed on screen 704. The interactive instructions may comprise a feature-rich presentation including a multimedia audio/video recording providing visual and audio instructions for operating the device, or may simply be a text file, displayed on screen 704, illustrating step-by-step instructions for operating and using the device. The inclusion of interactive instructions with the device eliminates the need for a device that requires extensive training to use, allowing for deployment and use by non-medical professionals.

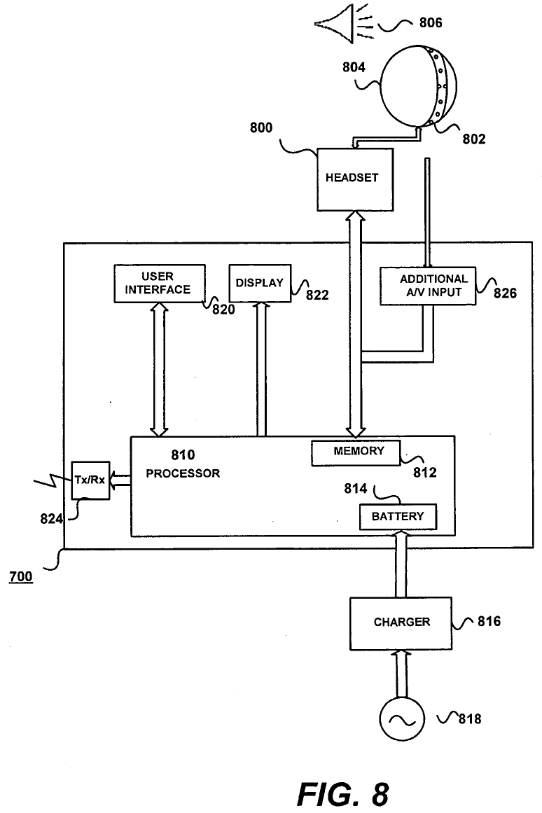

FIG. 8 shows a schematic diagram of portable handheld base unit 700 consistent with the present invention. As shown in FIG. 8, handheld base unit 700 is connected to headset 800. Headset 800 may include an electrode set 802 for detecting brain electrical signals to be placed on a subject's head 804. Electrode set 802 may comprise a reduced electrode set, having less than 19 electrodes, and preferably less than 10 electrodes. Headset 800 may also include a stimulus emitter 806 to be used for evoked potential tests. Stimulus emitter 806 may include an audio or visual stimulus emitter. The headset may also include analog amplification channels connected to the electrodes, and an analog-to-digital converter to digitize the acquired brain electrical signals prior to transmission to the handheld base unit 700.

Handheld base unit 700 also includes an electronics block 808 including processor 810, memory 812, and a power source 814 for providing power to the electronics block. In one embodiment consistent with the present invention, power source 814 comprises a rechargeable battery, which can be recharged when coupled to a charger 816 being powered by an AC or DC power source 818.

Electronics block 808 is further coupled to headset 800, user interface electronics 820 for controlling, for example, navigational pad 702, display electronics 822 for controlling, for example, screen 704, and consistent with an embodiment of the present invention, wireless electronics 824 for controlling, for example, wireless transceiver 708 and/or a wireless connection 706 to headset 800. In general, memory 812 contains instructions for causing processor 810 to perform functions for operating portable handheld device 700, including all of the electronics illustrated in FIG. 8, and for performing tests on a subject and providing a diagnosis based on the performed tests, consistent with the diffusion geometric analysis algorithms described herein, and in FIG. 1.

In accordance with the diffusion geometry algorithms of the Bx™ technology, the diffusion coordinates space may be divided into regions representing certain neurological states. The neurological states may be indicative of whether a subject is exhibiting normal or abnormal brain function. Moreover, abnormal brain function may be further broken down into diagnostic categories, which are indicative of conditions that are psychiatric or “functional” in nature, organic in nature, either lateral or global, or an emergency or “Alert” condition, which may include seizure, abnormal brainstem response, or burst suppression. Psychiatric or “functional” brain condition may further be broken down into specific diagnostic categories indicative of specific types of psychiatric disorders. Similarly, organic lateral and global brain functions may further be broken down into specific diagnostic categories indicative of specific types of lateral and global abnormalities. The ability of the methods and apparatus disclosed herein to determine a probability that a subject is experiencing a particular type of abnormal brain function, or the probability of a subject transitioning from a normal brain function to an abnormal brain function, allows a medical professional to act accordingly. For example, should a subject be diagnosed as having a high probability of having a brain function that is indicative of an organic abnormality, the apparatus will further determine whether the brain function has a higher probability of being indicative of a lateral or global abnormality, allowing a medical professional to distinguish between global abnormalities, such as concussion, toxicity, encephalitis and the like, and lateral abnormalities, such as ischemic and hemorrhagic strokes. This probability that a subject belongs to a particular diagnostic category can be displayed on display 222, which is operatively coupled to the processor. The display may be contained within the handheld base unit, or may be configured as a remote unit wirelessly connected to the processor in accordance with the Bx™ technology.

FIG. 9 shows an electrode set 802 consistent with an embodiment of the present invention. Electrode set 802 may comprise a plurality of electrodes, which may be affixed to the head of a subject 804. In an illustrative embodiment consistent with the Bx™ technology, electrode set 802 comprises nine electrodes that may be affixed to the forehead, shoulder and ear of the subject. This reduced electrode set 802 allows for placement on the forehead, and eliminates the need to place any electrodes over any hair that a subject may have on their head. This further eliminates any conduction problems that arise due to the hair, and also eliminates the need for any hair removal. In an illustrative embodiment, the electrodes may be placed on the right mastoid 902, far right of the forehead 904, near right of the forehead 906, center top of the forehead 908, near left of the forehead 910, far left of the forehead 912, left mastoid 914, and an ECG electrode on the left shoulder 916. Additionally, in an illustrative embodiment, there is an electrode placed on the center of the forehead 918 that is grounded. The electrodes on the right and left mastoids 902, 914 and the center of the forehead 918 may be used in an embodiment wherein an AEP (Auditory Evoked Potential) signal is acquired. An illustrative embodiment consistent with the present invention is able to use an electrode set 802 with a reduced number of electrodes, because the signal processing algorithms executed by processor 810 eliminate the need for additional electrodes.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of determining a neurological state of subject comprising the steps of:
acquiring electrical signals from the brain using at least one electrode channel;
extracting features from the acquired signals; and
classifying the extracted features into one or more diagnostic categories;
wherein the steps of extracting features and classifying extracted features is performed using diffusion geometric analysis.

2. The method of claim 1, further comprising the steps of:
amplifying the acquired brain electrical signals;
digitizing the amplified brain electrical signals to obtain a digital data set.

3. The method of claim 1, wherein the electrical signals from the brain comprises spontaneous electrical activity.

4. The method of claim 1, wherein the electrical signals from the brain comprises evoked potentials.

5. The method of claim 1, wherein the electrical signals from the brain comprises spontaneous electrical activity and evoked potentials.

6. The method of claim 1, wherein the step of extracting features comprises the steps of:
organizing an acquired data set into a collection of digital documents, wherein each document comprises a time window of features of the measurement of each electrode channel;
determining a left and a right singular vector for each digital document;
constructing an affinity matrix using the left or right singular vector to determine affinity between at least two digital documents of the data set;
determining the eigenvectors of the affinity matrix to compute a diffusion geometry of the data set comprising at least a plurality of diffusion coordinates;
embedding of the data into the first three diffusion coordinates to generate a diffusion map, wherein diffusion distances are encoded as Euclidean distances.

7. The method of claim 6, wherein a time-stamped digital document is an M×L matrix, wherein M is the number of electrode channels and L is the length of the time window.

8. The method of claim 6, wherein the features comprise temporal features.

9. The method of claim 8, wherein the temporal features are wavelet packet features.

10. The method of claim 6, wherein the features comprise spectral features.

11. The method of claim 6, wherein the affinity matrix is a bi Markov matrix.

12. The method of claim 6, further comprising the step of configuring a measurement space through the diffusion distances in order to extract a tabulation of measurement states and their temporal dynamics.

13. The method of claim 6, wherein the diffusion coordinates are stored in a database.

14. The method of claim 13, wherein the stored diffusion coordinates of a plurality of data sets are used as inputs in a diagnostic test.

15. The method of claim 6, wherein classifying the data set comprises partitioning predetermined spaces in the diffusion coordinates space into partitions corresponding to particular neurological states.

16. The method of claim 15, further comprising the step of determining the probability of transition between classified states.

17. The method according to claim 1, further comprising a step of:
determining a neurological state of the subject based on a classification.

18. The method of claim 1, further comprising a step of denoising the acquired signals by computing a diffusion geometry of a data set corresponding to the acquired signal, wherein the diffusion geometry comprises at least a plurality of diffusion coordinates.

19. The method of claim 18, wherein an affinity matrix is used to define non-linear filters for denoising and artifact detection.

20. The method of claim 19, further comprising a step of subtracting detected artifacts to obtain a clean brain electrical signal.

21. The method of claim 19, wherein denoising further comprises:
describing a particular feature of the data set as a function;
filtering the function using the affinity matrix to produce a filtered function;
determining diffusion distances between the function and the filtered function; and
extracting particular features wherein the diffusion distance is greater than a pre-determined threshold.

22. A method of classifying a neurological state of subject using diffusion geometry of a data set.

23. The method of claim 22, wherein classifying the neurological state comprises the step of comparing the data set to another data set based on diffusion geometry associated with each dataset.

24. An apparatus for signal processing, comprising:
a signal receiver unit or a headset;
a handheld base unit, wherein
a processor is configured to utilize one or more operating instructions stored in a memory to perform denoising of the received signal, extract features, and classify the signal using diffusion geometric analysis;
a display unit, wherein a result from the processor is displayed.

25. The apparatus of claim 24, wherein the said signal receiver unit comprises at least one electrode, at least one analog amplification channel, and an analog-to-digital converter.

26. The apparatus of claim 24, wherein the signal receiver unit and the handheld base unit are directly connected.

27. The apparatus of claim 24, wherein the signal receiver unit communicates wirelessly with the handheld base unit.

28. The apparatus of claim 27, wherein the signal receiver unit further comprises a wireless transmitter.

29. The apparatus of claim 27, wherein the handheld base unit further comprises a wireless receiver.

30. The apparatus of claim 24, wherein the display unit is operatively connected to the processor to display results of one or more operations performed by the processor; and
wherein the display unit can be integrated into the handheld base unit, or can be external to the handheld base unit.

31. The apparatus of claim 24, wherein the signal receiver unit and the handheld base unit may be included in a single kit for field use or point-of-care applications.

32. The apparatus of claim 25, wherein the signal receiver unit and the handheld base unit may be configured to reside on a common platform; and
wherein the at least one analog amplification channel, the analog-to-digital converter, and the processor are integrated into a single chip.

33. The apparatus of claim 24, wherein the processor is configured to compute diffusion geometry of a data set comprising at least a plurality of diffusion coordinates.

34. The apparatus of claim 33, wherein the memory is configured to store the diffusion coordinates.
